(19) United States
(12) Patent Application Publication
(10) Pub. No.: US 2022/0287876 A1
(43) Pub. Date: Sep. 15, 2022

(54) Thermoelectric Heating/Cooling Pad

(71) Applicant: C. R. Bard, Inc., Franklin Lakes, NJ (US)

(72) Inventors: Jesse Smith, Broomfield, CO (US); Mengjia Yi, Westminster, CO (US); Niharika Pathare, Thane (IN); Gabriel A. Johnston, Broomfield, CO (US)

(21) Appl. No.: 17/690,908

(22) Filed: Mar. 9, 2022

Related U.S. Application Data
(60) Provisional application No. 63/159,147, filed on Mar. 10, 2021.

(51) Int. Cl. A61F 7/02

(52) U.S. Cl. CPC A61F 7/02; A61F 2007/0246; A61F 2007/0076

(57) ABSTRACT

Disclosed are systems and methods for performing targeted temperature management including a thermal pad having a thermoelectric device. A pad controller may regulate the temperature of the thermal pad. The thermal pad temperature can facilitate cooling and/or warming of the patient. The pad can include a thermal conduction layer and a skin contact layer. The system may include a controller to set the pad temperature of multiple thermal pads according to a TTM therapy. The thermal pad and the system can incorporate data from temperature sensors coupled to the thermoelectric device and the patient. The system controller may be coupled to the thermal pads via a wired or wireless connection.

### PRIORITY

This application claims the benefit of priority to U.S. Provisional Application No. 63/159,147, filed Mar. 10, 2021, which is incorporated by reference in its entirety into this application.

### BACKGROUND

The effect of temperature on the human body has been well documented and the use of targeted temperature management (TTM) systems for selectively cooling and/or heating bodily tissue is known. Elevated temperatures, or hyperthermia, may be harmful to the brain under normal conditions, and even more importantly, during periods of physical stress, such as illness or surgery. Conversely, lower body temperatures, or mild hypothermia, may offer some degree of neuroprotection. Moderate to severe hypothermia tends to be more detrimental to the body, particularly the cardiovascular system.

Targeted temperature management can be viewed in two different aspects. The first aspect of temperature management includes treating abnormal body temperatures, i.e., cooling the body under conditions of hyperthermia or warming the body under conditions of hypothermia. The second aspect of thermoregulation is an evolving treatment that employs techniques that physically control a patient's temperature to provide a physiological benefit, such as cooling a stroke patient to gain some degree of neuroprotection. By way of example, TTM systems may be utilized in early stroke therapy to reduce neurological damage incurred by stroke and head trauma patients. Additional applications include selective patient heating/cooling during surgical procedures such as cardiopulmonary bypass operations.

Some TTM systems circulate a fluid (e.g., water) through one or more thermal contact pads coupled to a patient to affect surface-to-surface thermal energy exchange with the patient. In general, TTM systems comprise a TTM fluid control module coupled to at least one contact pad via a fluid deliver line. Such TTM systems include expensive capital equipment that requires maintenance. Fluid leaks may take place causing exposure of the patient to the fluid directly.

In summary, there is a need for a TTM system that does not include either TTM fluid or the capital equipment necessary for TTM fluid preparation and handling.

### SUMMARY OF THE INVENTION

Briefly summarized, disclosed herein is a medical pad for providing a targeted temperature management (TTM) therapy to a patient. The pad may comprise at least one thermoelectric device (TED) having a top side and a bottom side, the TED configured to establish a temperature difference between the top side and the bottom side in accordance with electrical energy supplied to the TED. The pad further includes a thermal conduction layer coupled to the bottom side and an insulation layer coupled to the thermal conduction layer. The pad is configured to exchange thermal energy with the patient according to a temperature difference between the bottom-side temperature and a temperature of the patient. In some embodiments, the pad may be configured to extract thermal energy away from the patient.

The pad may comprise a thermal conduction gel disposed across at least a portion of a top side and/or a bottom side of the thermal conduction layer. The pad may further comprise a convection device coupled to the top side of the TED and the convection device is configured to facilitate thermal energy exchange between the TED and environmental air adjacent the top side of the TED. The pad may further comprise a fan to provide airflow across the convection device.

The pad may comprise a pad controller configured to regulate a pad temperature, where the pad temperature is defined at least partially by the bottom-side temperature of the TED. The controller is electrically coupled to the TED and the controller is configured to regulate the electrical energy supplied to the TED.

The pad may further comprise a temperature sensor operatively coupled to the bottom side of the TED. The temperature sensor is also coupled to the controller and the controller is configured to regulate the pad temperature as measured by the temperature sensor.

The pad may further comprise a plurality of TEDs coupled to the conduction layer and the controller may be configured to regulate the electrical energy supplied to the plurality of TEDs.

The pad may further comprise a hinge portion extending between adjacent TEDs. The hinge portion is configured to facilitate pivotable displacement of each TED with respect to an adjacent TED.

The pad may comprise two or more temperature zones with each zone comprising one or more TEDs and the controller is configured to independently regulate the pad temperature of each temperature zone. The pad may comprise a plurality of controllers, where each controller is configured to regulate the electrical energy supplied to a single TED or a subset of the plurality of TEDs.

In some embodiments, the pad further includes a top side temperature sensor operatively coupled to the top side of the TED, where the top side temperature sensor is coupled to the controller, the fan is coupled to the controller, and the controller is configured to adjust electrical power supplied to the fan in order to regulate a top side temperature of the TED as measured by the top side temperature sensor.

In some embodiments, the pad further includes a circulating liquid system configured to transfer thermal energy away from the top side of the TED. The liquid system includes a heat exchanger coupled to the top side and a radiator coupled to the heat exchanger via a liquid conduit loop, where the loop provides a delivery liquid flow path from the heat exchanger to the radiator and a return liquid flow path radiator to the heat exchanger. A pump is disposed in line with the liquid conduit loop to define liquid flow through the loop, and the fan is coupled to the radiator to provide air flow through the radiator.

Disclosed herein also is a targeted temperature management (TTM) system including a thermal pad that comprises a thermoelectric device (TED) having a top side and a bottom side, where the TED is configured to establish a temperature difference between the top side and the bottom side in accordance with electrical energy supplied to the TED. The thermal pad further includes a pad controller coupled to the TED, where the pad controller is configured to regulate a pad temperature of the thermal pad according to a target pad temperature stored in memory on the pad controller, and the pad temperature is defined at least partially by a bottom-side temperature of the TED. The TTM system also comprises a system controller coupled to the thermal pad and the system controller is configured to define the target pad temperature of the thermal pad.

The system controller may be coupled to the thermal pads via a wireless connection. The system may further comprise a patient temperature sensor configured to measure a temperature of the patient and the patient temperature sensor is coupled to the system controller. The system controller is configured to define the target pad temperature according to a target patient temperature stored in memory on the system controller.

The system may comprise a plurality of thermal pads and the system controller may be configured to define the target pad temperature for each of the plurality of thermal pads. In some embodiments, the target pad temperature for one of the plurality of thermal pads may be different from the target pad temperature for another of the plurality of thermal pads.

Disclosed herein is also a method of providing a targeted temperature management (TTM) therapy to a patient. The method includes providing a thermal pad including at least one thermoelectric device coupled to a controller. The method further includes applying the thermal pad to the patient, coupling the thermal pad to an electrical power source, and exchanging thermal energy between the patient and environmental air adjacent the pad, where the thermal energy passes through the pad.

The method may further comprise controlling a thermal pad temperature at a target pad temperature stored in memory on the controller. In some embodiments, the target pad temperature is lower than a temperature of the patient.

In some embodiments of the method, the pad comprises a first temperature zone and a second temperature zone, and controlling a thermal pad temperature comprises controlling a first thermal pad temperature of the first temperature zone, and controlling a second thermal pad temperature of the second temperature zone, where the second thermal pad temperature is different from the first thermal pad temperature.

In some embodiments of the method, the thermal pad comprises a patient temperature sensor coupled to the controller, and controlling a thermal pad temperature comprises defining target pad temperature to achieve a target patient temperature stored in memory on the controller.

These and other features of the concepts provided herein will become more apparent to those of skill in the art in view of the accompanying drawings and the following description, which describe particular embodiments of such concepts in greater detail.

### BRIEF DESCRIPTION OF DRAWINGS

A more particular description of the present disclosure will be rendered by reference to specific embodiments thereof that are illustrated in the appended drawings. It is appreciated that these drawings depict only typical embodiments of the invention and are therefore not to be considered limiting of its scope. Example embodiments of the invention will be described and explained with additional specificity and detail through the use of the accompanying drawings in which:

FIG. 1 is an illustration of a thermal pad in use within a targeted temperature management (TTM) environment, in accordance with some embodiments.

FIG. 2A is a detail cross-sectional side view of a portion of the thermal pad of FIG. 1, in accordance with some embodiments.

FIG. 2B is a detail cross-sectional side view of an edge portion of the thermal pad of FIG. 1, in accordance with some embodiments.

FIG. 3 illustrates a block diagram of a controller of the thermal pad for FIG. 1, in accordance with some embodiments.

FIG. 4 is a top view illustration of a second embodiment of a thermal pad, in accordance with some embodiments.

FIG. 5 is a top view illustration of a third embodiment of a thermal pad, in accordance with some embodiments.

FIG. 6 is an illustration of a TTM system in use within targeted temperature management (TTM) environment, in accordance with some embodiments.

FIG. 7 illustrates a block diagram of a console of the system controller of FIG. 6, in accordance with some embodiments.

FIG. 8 illustrates an embodiment of the thermal pad incorporating natural convective transfer of thermal energy away from the thermoelectric device, in accordance with some embodiments.

FIG. 9 illustrates an embodiment of the thermal pad incorporating a radiator with a circulating liquid to transfer thermal energy away from the thermoelectric device, in accordance with some embodiments.

### DETAILED DESCRIPTION

Before some particular embodiments are disclosed in greater detail, it should be understood that the particular embodiments disclosed herein do not limit the scope of the concepts provided herein. It should also be understood that a particular embodiment disclosed herein can have features that can be readily separated from the particular embodiment and optionally combined with or substituted for features of any of a number of other embodiments disclosed herein.

Regarding terms used herein, it should also be understood the terms are for the purpose of describing some particular embodiments, and the terms do not limit the scope of the concepts provided herein. Ordinal numbers (e.g., first, second, third, etc.) are generally used to distinguish or identify different features or steps in a group of features or steps, and do not supply a serial or numerical limitation. For example, “first,” “second,” and “third” features or steps need not necessarily appear in that order, and the particular embodiments including such features or steps need not necessarily be limited to the three features or steps. Labels such as “left,” “right,” “top,” “bottom,” “front,” “back,” and the like are used for convenience and are not intended to imply, for example, any particular fixed location, orientation, or direction. Instead, such labels are used to reflect, for example, relative location, orientation, or directions. Singular forms of “a,” “an,” and “the” include plural references unless the context clearly dictates otherwise. The words “including,” “has,” and “having,” as used herein, including the claims, shall have the same meaning as the word “comprising.” Furthermore, the terms “or” and “and/or” as used herein are to be interpreted as inclusive or meaning any one or any combination. As an example, “A, B or C” or “A, B and/or C” mean “any of the following: A; B; C; A and B; A and C; B and C; A, B and C.” An exception to this definition will occur only when a combination of elements, components, functions, steps or acts are in some way inherently mutually exclusive.

The phrases “connected to” and “coupled to” refer to any form of interaction between two or more entities, including mechanical, electrical, magnetic, electromagnetic, fluid, signal, communicative (including wireless), and thermal interaction. Two components may be connected or coupled to each other even though they are not in direct contact with each other. For example, two components may be coupled to each other through an intermediate component.

Unless defined otherwise, all technical and scientific terms used herein have the same meaning as commonly understood by those of ordinary skill in the art.

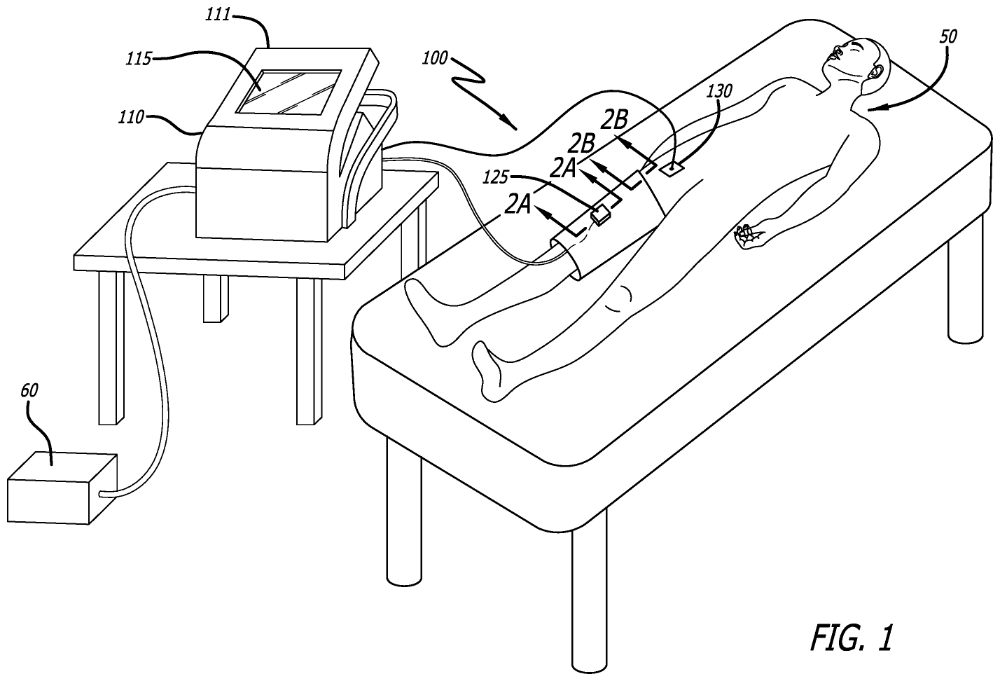

FIG. 1 is perspective view illustration of a thermal pad 100 in use in targeted temperature management (TTM) environment, in accordance with some embodiments. FIG. 1 shows a thermal pad 100 applied to a patient 50 for the administration of a (TTM) therapy to the patient 50. The TTM therapy may include cooling and/or warming of the patient 50, in accordance with some embodiments. The thermal pad 100 may be coupled to a pad controller 110 that includes a housing 111 and an operator interface 115 such as a graphical user interface (GUI). In some embodiments, the operator interface 115 may be disposed within the housing 111. The thermal pad 100 is coupled to an electrical power source 60. In some embodiments, the power source 60 may be an electrical power system of a facility. In other embodiments, the power source 60 may be a portable power source such as a battery pack or a generator. The thermal pad 100 converts electrical power from the power source 60 into thermal energy exchange with the patient 50 via a thermoelectric device (TED) 125. The thermal exchange is facilitated via application of the thermal pad 100 to the skin of the patient 50.

In use, the pad controller 110 may continually control the thermal energy exchange with the patient 50 to obtain a core or localized temperature of the patient 50 in accordance with the prescribed TTM therapy. In some embodiments, the thermal pad 100 may be coupled to a body temperature sensor 130. The body temperature sensor 130 is coupled to the patient 50 to indicate a core body temperature of the patient 50 in accordance with the prescribed TTM therapy. In some instances, the body temperature sensor 130 may be an esophageal, bladder catheter, or rectal temperature sensor. In use, the pad controller 110 may acquire body temperature data from the body temperature sensor 130 and establish thermal pad temperature to move the body temperature of the patient toward a target body temperature. In other words, the pad controller 110 may regulate the thermal pad temperature according to the TTM therapy. In some embodiments, the pad controller 110 may be embedded within the thermal pad 100.

The operator interface 115 may facilitate operation of the thermal pad 100 by a clinician according to a prescribed TTM therapy. By way of the operator interface 115, the clinician may set a target body temperature of the patient, a target pad temperature, and other parameters that may apply to the TTM therapy. In some instances, the target body temperature may change during the TTM therapy according to a temperature profile defined by the prescribed TTM therapy. The operator interface 115 may also display operating parameters of the TTM therapy such as the current body temperature and the current pad temperature, for example. In some embodiments, the operator interface 115 may be wirelessly coupled to the controller. For example, the operator interface 115 may comprise a software application running on an external device such as a network computer, a tablet, or a cell phone.

As illustrated in FIG. 1, an embodiment of the TED 125 may comprise a rectangular shape when viewed from the top. In other embodiments, the TED 125 may comprise the shape of a triangle, a square, or any other regular or non-regular polygon. In some embodiments, the TED may be flexible and/or curved.

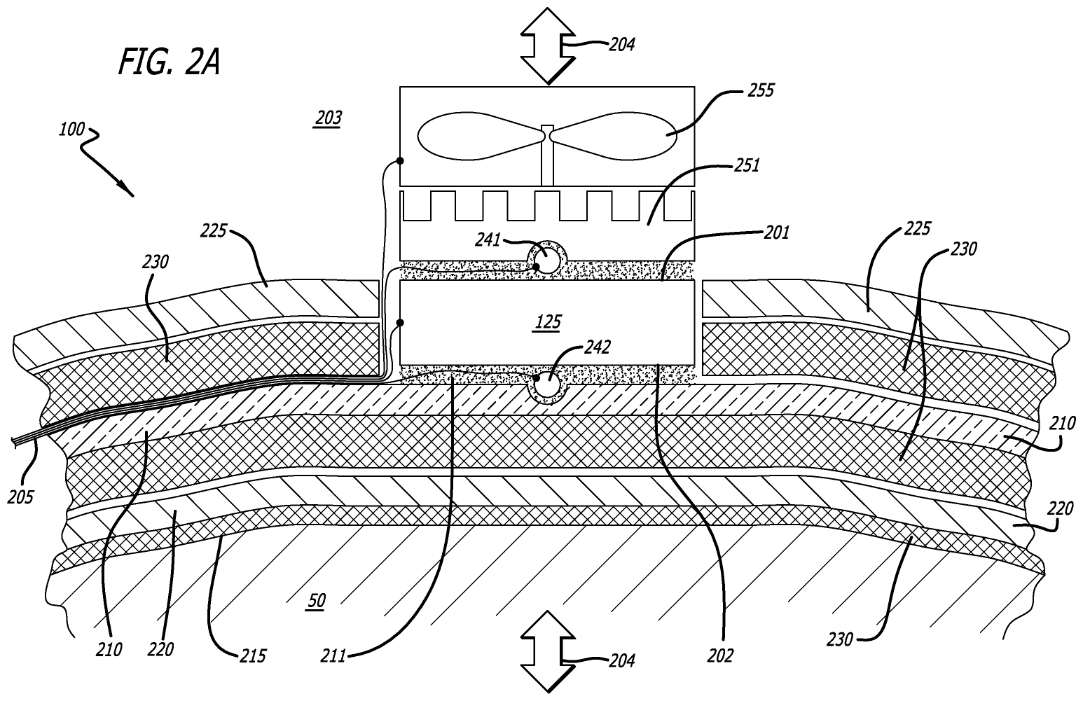

FIG. 2A illustrates a detailed cross-sectional side view of a portion of the thermal pad 100 cut along sectioning lines 2A-2A. As shown, the TED 125 includes a top side 201 disposed toward the environment 203 away from the patient 50 and a bottom side 202 disposed toward the patient 50. As illustrated in FIG. 2A, the bottom side 202 may include a planar surface. In other embodiments, the bottom side 202 may include a one-directional curved surface or a two-directional curved surface. In some embodiments, the TED 125 may comprise an electrically insulative coating (not shown), disposed on the top side 201 and/or the bottom side 202. In some embodiments, the coating may extend around a circumference of the TED 125. The coating may include thermal-conductive properties. The TED 125 is configured to define a temperature difference between the top side 201 and the bottom side 202 in accordance with a direct-current (DC) voltage supplied to the TED 125. The magnitude of temperature difference between the top side 201 and a bottom side 202 is defined by the magnitude of the DC voltage supplied to the TED 125 and direction of the temperature difference is defined by the polarity of the DC voltage. The polarity of the DC voltage as defined by the pad controller 110 may define a temperature of the top side 201 that is hotter than the bottom side 202. Conversely, a reversed polarity of the DC voltage as defined by the pad controller 110 may define a temperature of the top side 201 that is colder than the bottom side 202. In use, the pad controller supplies electrical energy to the TED 125 to establish the temperature difference between the top side 201 and a bottom side 202 and the temperature difference causes thermal energy exchange between the top side 201 and a bottom side 202.

By way of example, the pad controller 110 may supply electrical energy to the TED 125 to establish a temperature difference between the top side 201 and a bottom side 202 such that the temperature of the bottom side 202 (i.e., the thermal pad temperature) is less than the temperature of the patient 50 and the temperature of the top side 201 is greater than the temperature of the environment 203 (i.e., the environmental air adjacent the top side 201). In this example, thermal energy 204 is drawn away from the patient 50 toward the bottom side 202 of the TED 125 thereby cooling the patient 50. The thermal energy 204 then passes through the TED 125 from the bottom side 202 to the top side 201. The thermal energy 204 is finally drawn away from the top side 201 of the TED 125 by the environment 203. By way of summary, thermal energy is exchanged between the patient 50 and the environmental air adjacent a top side of the thermal pad 100.

By way of an alternative example, the pad controller 110 may supply electrical energy to the TED 125 to establish a temperature difference between the top side 201 and a bottom side 202 such that the temperature of the bottom side 202 (i.e., the thermal pad temperature) is greater than the temperature of the patient 50 and the temperature of the top side 201 is less than the temperature of the environment 203 (i.e., the environmental air adjacent the top side 201). In this example, thermal energy 204 is drawn from the environment 203 toward the top side 202 of the TED 125. The thermal energy then passes through the TED 125 from the top side 201 to the bottom side 202. The thermal energy 204 is finally drawn away from the bottom side 202 of the TED 125 by the patient 50 thereby warming the patient 50.

The thermal pad 100 may comprise one or more layers disposed between the bottom side 202 and the patient 50. The bottom side 202 may be coupled to a thermal conduction layer 210 via a thermally conductive adhesive 211. The thermal conduction layer 210 facilitates thermal energy exchange between the bottom side 202 and the patient 50. The thermal conduction layer 210 may also facilitate lateral conduction of thermal energy with respect to the TED 125 (i.e., along the thermal conduction layer 210) to define a uniform temperature of the thermal conduction layer 210. The thermal conduction layer 210 may be formed of a metalized film (e.g., one or more layers of copper, aluminum and/or brass). The thermal conduction layer 210 may be flexible so that the thermal pad 100 may acquire a curved shape when applied to the patient 50.

The thermal pad 100 may comprise a thermal conduction gel 230 disposed along a top surface and/or a bottom surface of the thermal conduction layer 210. The thermal conduction gel 230 may be comprised of an adhesive cross-linked hydrogel material such as is described in U.S. Pat. No. 5,645,855 to Lorenz. The thermal conduction gel 230 may or may not be disposed between the TED 125 and the thermal conduction layer 210. The thermal conduction gel 230 may further facilitate lateral conduction of thermal energy with respect to the TED 125. The lateral conduction of thermal energy provided by the thermal conduction gel 230 may combine with the lateral conduction of thermal energy provided by thermal conduction layer 210 to provide a consistent temperature along the thermal conduction layer 210. In some embodiments, the thermal conduction gel 230 disposed along the top surface and/or the bottom surface of the thermal conduction layer 210 may be omitted.

The thermal pad 100 may comprise a skin contact layer 220 coupled to a bottom side of the thermal conduction layer 210, such that in use, the skin contact layer 220 is disposed between the thermal conduction layer 210 and the patient 50. The skin contact layer 220 may comprise a foam structure or any other structure suitable for comfortable contact with the skin and thermal energy exchange between the thermal conduction layer 210 and the patient 50. The skin contact layer 220 may enhance thermal comfort to the patient during use of the thermal pad 100. For example, the skin contact layer 220 may include a balance between thermal conduction and thermal insulation properties to reduce patient discomfort (e.g., burning) that may be associated with hot spots of the thermal conduction layer 210, while maintaining thermal energy exchange between the thermal conduction layer 210 and the patient 50. The thermal conduction properties of the skin contact layer 220 may also further promote a consistent temperature along the thermal conduction layer 210. The skin contact layer 220 in combination with the thermal conduction layer 210 may provide for a uniform temperature along a patient contact surface 215 of the thermal pad 100 and thereby provide a uniform thermal energy exchange with the patient 50 along the patient contact surface 215. The skin contact layer 220 may be flexible so that the thermal pad 100 may acquire a curved shape when applied to the patient 50. The skin contact layer 220 may also include electrical insulation properties to electrically isolate the patient 50 from the TED 125.

In the illustrated embodiment, the thermal conduction gel 230 may also be disposed along a bottom side 215 of the skin contact layer 220, so as to be disposed between the skin contact layer 220 and the patient 50 when the pad 100 is applied to the patient 50. The thermal conduction gel 230 on the bottom side 215 may provide for constant uniform contact between the pad 100 the patient 50 and thereby promote constant uniform thermal energy exchange with the patient 50 across the pad 100. In other embodiments, disposition of the thermal conduction gel 230 along a bottom side 215 of the skin contact layer 220 may be omitted.

In another embodiment, the skin contact layer 220 may be formed of layer of a plurality of closed, fluid-filled pockets (not shown). The pockets may be configured to promote convective current flow of the fluid within the pockets and thereby facilitate convective heat transfer between the thermal conduction layer 210 and the patient 50. Such an embodiment may further promote lateral disbursement of temperature across the pad 100.

In some embodiments, the skin contact layer 220 may be separable from the pad 100. In such embodiments, the skin contact layer 220 may be separated from the pad after use and replaced with another (e.g., new) skin contact layer 220 prior to a subsequent use of the pad 100. Such embodiments may allow the pad 100 to be used multiple times across the same patient or different patients while providing a new skin contact interface with each application of the pad 100.

In some embodiments, the thermal pad 100 may include an upper insulation layer 225 disposed over the thermal conduction gel 230. The upper insulation layer 225 may be configured to inhibit thermal energy exchange between the thermal conduction gel 230 and the environment 203. The upper insulation layer 225 may be formed of a foam structure or any other structure consistent with thermal insulation. The upper insulation layer 225 may comprise an opening for each TED 125 so that the upper insulation layer 225 does not inhibit thermal energy exchange between the top side 201 of the TED 125 and the environment 203. The upper insulation layer 225 may be flexible so that the thermal pad 100 may acquire a curved shape when applied to the patient 50. In some embodiments, the upper insulation layer 225 may be omitted.

The thermal pad 100 may also comprise top-side temperature sensor 241. The top-side temperature sensor 241 is operatively coupled to the top side 201 of the TED 125 to measure the temperature of the top side 201. The top-side temperature sensor 241 may be coupled to the pad controller 110 to provide temperature data to the pad controller 110 as further described below.

The thermal pad 100 may comprise bottom-side temperature sensor 242. The bottom-side temperature sensor 242 is operatively coupled to the bottom side 202 of the TED 125 to measure the temperature of the bottom side 202 (i.e., the thermal pad temperature). The bottom-side temperature sensor 242 may be coupled to the pad controller 110 to provide temperature data to the pad controller 110 as further described below.

The thermal pad 100 may comprise a thermal convection device 251 to promote thermal energy exchange between the top side 201 of the TED 125 and the environment 203. The thermal convection device 251 may be thermally coupled to the top side 201 via a thermally conductive adhesive, grease, pad or any another thermally conduction medium. In some embodiments, the thermal convection device 251 may be a heat sink. The thermal convection device 251 may be configured to exchange thermal energy with the environment 203 via natural convection or forced convection.

In some embodiments, the thermal pad 100 may comprise a fan 255 coupled to the thermal pad 100 to provide forced convection airflow with respect to the thermal convection device 251. In some embodiments, the fan 255 may be attached to the TED 125. The fan 255 may be coupled to and controlled by the pad controller 110. In other embodiments, the fan 255 may be omitted.

In some embodiments, electrical wires 205 extending away from the TED 125, the top-side temperature sensor 241, bottom-side temperature sensor 242, and/or the fan 255 may be embedded within the thermal pad 100. In other words, the wires 205 may be disposed between the bottom side 215 of the skin contact layer 220 and a top side of the upper insulation layer 225. The wires 205 may be disposed between adjacent layers of the thermal pad 100 or disposed within a layer of the thermal pad 100 such as the skin contact layer 220, the thermal conduction gel 230, or the upper insulation layer 225. In some embodiments, the wires 205 may exit the thermal pad 100 at a perimeter of the thermal pad 100.

FIG. 2B is a detailed cross-sectional side view of an edge portion of the thermal pad of FIG. 1 cut along sectioning lines 2B-2B, in accordance with some embodiments. In some embodiments, a circumference of the skin contact layer 220 may define a circumference of the thermal pad 100. In other words, a perimeter edge of the skin contact layer 220 may extend outwardly beyond perimeter edges of the thermal conduction layer 210, the thermal conduction gel 230, and the upper insulation layer 225. The thermal pad 100 may comprise a perimeter tape 235 extending along the circumference of the thermal pad 100. The perimeter tape 235 may be adhesively attached to the skin contact layer 220 and extend inwardly to cover the perimeter edges of any or all of the thermal conduction layer 210, the thermal conduction gel 230, and the upper insulation layer 225. In some embodiments, the upper insulation layer 225 and the skin contact layer 220 may be fused together along their circumferential edges to form a sealed pocket containing the thermally conductive gel 230. In such embodiments, the perimeter tape 235 may be omitted.

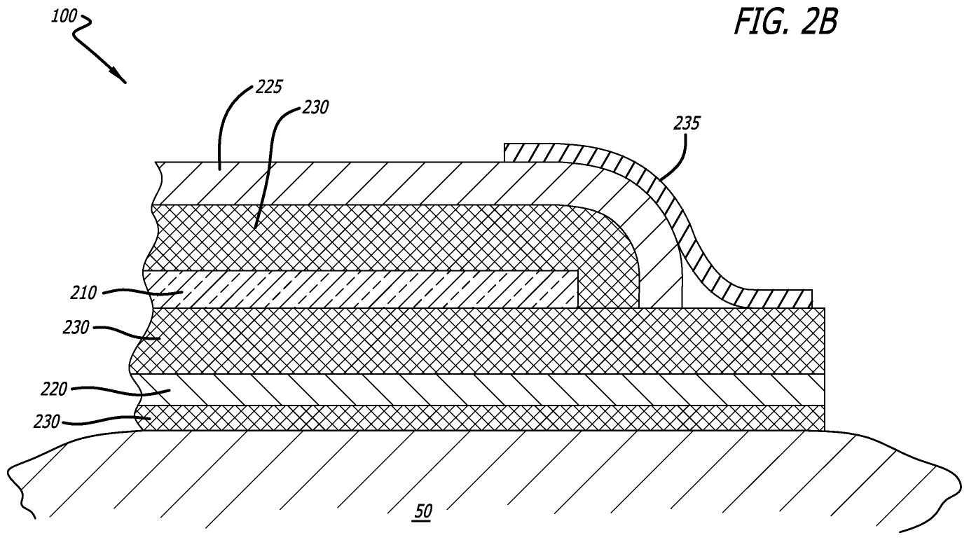

FIG. 3 illustrates a block diagram of the pad controller 110 of FIG. 1, in accordance with some embodiments. The pad controller 110 comprises a processor 305 and memory 310 including a non-transitory, computer-readable storage medium, and the memory 310 includes temperature control logic 315 stored thereon. The pad controller 110 further includes a power converter 320, a wireless module 317, and input/output (I/O) ports 330. The pad controller 110 receives electrical power from the power source 60. The I/O ports 330 facilitate connection of the pad controller 110 with the TED 125, the bottom-side temperature sensor 242, the top-side temperature sensor 241, the fan 255 and the body temperature sensor 130. The pad controller 110 may include or be coupled to the operator interface 115.

The power converter 320 converts electrical power from the power source 60 into a form of electrical power compatible with the TED 125. For example, the power source may provide electrical power in the form of alternating current (AC) at a relatively high voltage (e.g., 120 to 240 VAC). The power converter 320 may convert the in-coming high AC voltage to a reduced DC voltage. In some embodiments, the DC voltage may be less than about 24 VDC to operate the TED 125. The power converter 320 may also be configured to reverse the polarity of the DC voltage. In other words, the power converter 320 may provide a DC voltage between about +24 VDC and about −24 VDC. In other embodiments, the DC voltage may be more or less than about 24 VDC to operate the TED 125. The power converter 320 may be controllable via the temperature control logic 315 when executed by the processor 305.

The operator interface 115 is coupled to the pad controller 110 via a wired or wireless connection. In some embodiments, the operator interface 115 may be included with the pad controller 110. In other embodiments, the operator interface 115 may be operated via an external device such as a network computer, a tablet or a cell phone.

The wireless module 317 provides for wireless connection to devices external of pad controller 110. The external devices may include a facility network, other medical devices related to the TTM therapy, the operator interface 115, the body temperature sensor 130, or any other device that may be used in accordance with the TTM therapy.

The temperature control logic 315 is configured to control the temperature of the bottom side 202 of the TED 125 (i.e., the thermal pad temperature). The temperature control logic 315 may receive temperature data from the bottom-side temperature sensor 242 and adjust the DC voltage supplied to the TED 125 to move the temperature of the bottom side 202 of the TED 125, toward a target pad temperature stored in the memory 310. More specifically, the temperature control logic 315 may compare temperature data received from the bottom-side temperature sensor 242 with the target pad temperature. As a result of the comparison, the temperature control logic 315 as executed by the processor 305 may cause the power converter 320 to increase or decrease the DC voltage supplied to the TED 125 to move the thermal pad temperature toward the target pad temperature. By way of summary, the temperature control logic 315 is configured to establish and maintain the thermal pad temperature at the target pad temperature.

In some embodiments, the temperature control logic 315 may receive body temperature data from the body temperature sensor 130, and adjust the DC voltage supplied to the TED 125 to move the body temperature toward a target body temperature stored in the memory 310. More specifically, the temperature control logic 315 may compare temperature data received from the body temperature sensor 130 with the target body temperature. As a result of the comparison, the temperature control logic 315 as executed by the processor 305 may cause the power converter 320 to increase or decrease the DC voltage supplied to the TED 125 to move the temperature of the bottom side 202 of the TED 125 toward a pad temperature consistent with moving the body temperature toward the target body temperature stored in the memory 310.

In some embodiments, the temperature control logic 315 may compare temperature data received from the bottom-side temperature sensor 242 with a high thermal pad temperature limit stored in the memory 310. As a result of the comparison, the temperature control logic 315 as executed by the processor 305 may cause the power converter 320 to decrease the magnitude of DC voltage supplied to the TED 125 to move the thermal pad temperature away from the high thermal pad temperature limit, i.e., prevent the thermal pad temperature from exceeding the high thermal pad temperature limit. Preventing the thermal pad temperature from exceeding the high thermal pad temperature limit may reduce the risk of the pad 100 causing a burn to the patient 50.

In some embodiments, the temperature control logic 315 may compare temperature data received from the top-side temperature sensor 241 with a high top-side temperature limit stored in the memory 310. As a result of the comparison, the temperature control logic 315 as executed by the processor 305 may cause the power converter 320 to decrease the magnitude of DC voltage supplied to the TED 125 to move the top-side temperature away from the high top-side temperature limit, i.e., prevent the top side of the TED 125 from exceeding the high top-side temperature limit. Preventing the top side of the TED 125 from exceeding the high top-side temperature limit may reduce the risk of the TED 125 causing a burn to the patient 50. In some embodiments, as a result of the comparison, the temperature control logic 315 as executed by the processor 305 may cause the power converter 320 to supply electrical power to the fan 255 to move the top-side temperature away from the high top-side temperature limit.

In some embodiments, the temperature control logic 315 may calculate a temperature difference between temperature data received from the top-side temperature sensor 241 and temperature data received from the bottom-side temperature sensor 242. The temperature control logic 315 may then compare the calculated temperature difference with a target temperature difference stored in the memory 310. As a result of the comparison, the temperature control logic 315 as executed by the processor 305 may cause the power converter 320 to adjust the magnitude of DC voltage supplied to the TED 125 to move the temperature difference toward the target temperature difference.

In some embodiments, the temperature control logic 315 may be configured to control the temperature of the top side 201 of the TED 125 to establish or maintain a top-side temperature within a temperature range consistent with maximizing or optimizing an efficiency of the TED 125 or an efficiency of the thermal pad 100 as a whole. The temperature control logic 315 may receive temperature data from the top-side temperature sensor 241 and adjust an electrical power supplied to the fan 255 to move the temperature of the top side 201 of the TED 125 toward a target top-side temperature stored in the memory 310. More specifically, the temperature control logic 315 may compare temperature data received from the top-side temperature sensor 241 with the target top-side temperature. As a result of the comparison, the temperature control logic 315 as executed by the processor 305 may cause the power converter 320 to increase or decrease the electrical power supplied to the fan 255 to move the top-side temperature toward the target top-side temperature. By way of summary, the temperature control logic 315 may be configured to establish and maintain the top-side temperature at the target top-side temperature.

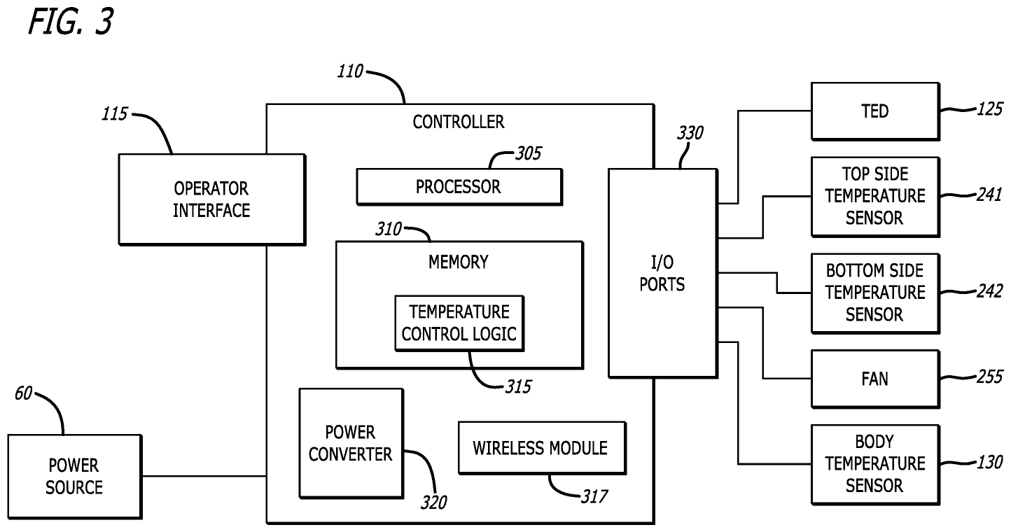

FIG. 4 illustrates a top view of another embodiment of the thermal pad of FIG. 1. The thermal pad 400 can, in certain respects, resemble components of the thermal pad 100 described in connection with FIGS. 1-3. It will be appreciated that all the illustrated embodiments may have analogous features. Accordingly, like features are designated with like reference numerals, with the leading digits incremented to “4.” For instance, the pad controller is designated as “110” in FIGS. 1-3, and an analogous pad controller is designated as “410” in FIG. 4. Relevant disclosure set forth above regarding similarly identified features thus may not be repeated hereafter. Moreover, specific features of the thermal pad 100 and related components shown in FIGS. 1-3 may not be shown or identified by a reference numeral in the drawings or specifically discussed in the written description that follows. However, such features may clearly be the same, or substantially the same, as features depicted in other embodiments and/or described with respect to such embodiments. Accordingly, the relevant descriptions of such features apply equally to the features of the thermal pad 400 of FIG. 4. Any suitable combination of the features, and variations of the same, described with respect to the thermal pad and components illustrated in FIGS. 1-3 can be employed with the thermal pad and components of FIG. 4, and vice versa. This pattern of disclosure applies equally to further embodiments depicted in subsequent figures and described hereafter.

The thermal pad 400 comprises two TEDs 425 coupled to a pad controller 410. However, the thermal pad 400 may include two, three, four or more TEDs 425. In some embodiments, the thermal pad 400 may comprise an array of TEDS 425 extending across an area of the thermal pad 400. The thermal pad 400 may comprise hinge portions 405 disposed between adjacent TEDs 425. The hinge portions 405 may for provide pivotable displacement of each TED 425 with respect to an adjacent TED 425 so that the thermal pad 400 may acquire a curved shape when applied to the patient 50. The hinge portions 405 may be defined by the flexibility of the thermal conduction layer 210, the insulation layer 210, and the upper insulation layer 230 as described above in relation to the FIG. 2A.

The pad controller 410 is configured to control the thermal energy exchange of both TEDs with the patient 50. The pad controller 410 may control the thermal energy exchange of each TED 425 individually or as a pair. In some instances, there may exist performance variation among multiple TEDs 425. For example, the temperature difference between a top side and a bottom side of the TEDs 425 may not be the same when the same DC voltage is applied to the TEDs 425. As such, the pad controller 410 may supply different levels of DC voltage to the TEDs 425 to obtain the same thermal pad temperature.

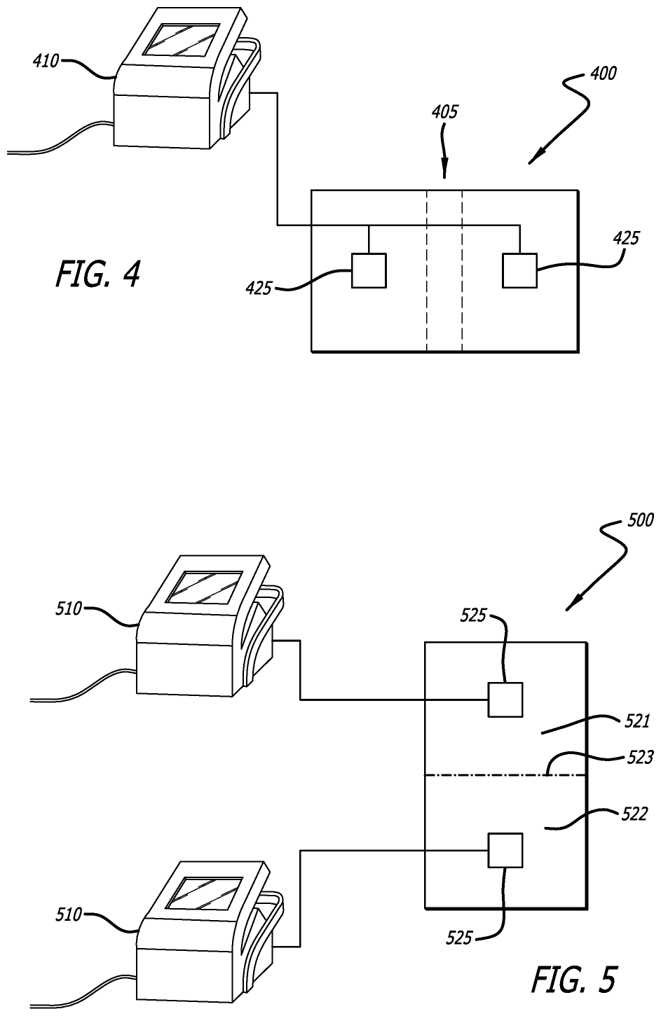

FIG. 5 illustrates a top view of another embodiment of the thermal pad of FIG. 1. The thermal pad 500 comprises two temperature zones 521, 522. Each zone 521, 522 comprises a TED 525 and a pad controller 510. In some embodiments, each zone 521, 522 may comprise one, two, three, four, or more TEDs 525 coupled to the respective pad controller 510. The thermal pad temperature for the zones 521, 522 may be the same or different. In other words, the target pad temperature may be the same or different for each zone 521, 522. As such, the thermal pad 500 may be used when the TTM therapy requires different target pad temperatures for the zones 521, 522. In some embodiments, the zones 521, 522 may be physically separable from each other along a separation line 523 to provide for additional placement options for the temperature zones 521, 522 on the patient 50. As each temperature zone 521, 522 comprises its own pad controller 510, operation of the temperature zones 521, 522 including enabling and disabling may be performed independently from each other. In other embodiments, the thermal pad 500 may include one, three, four, or more temperature zones.

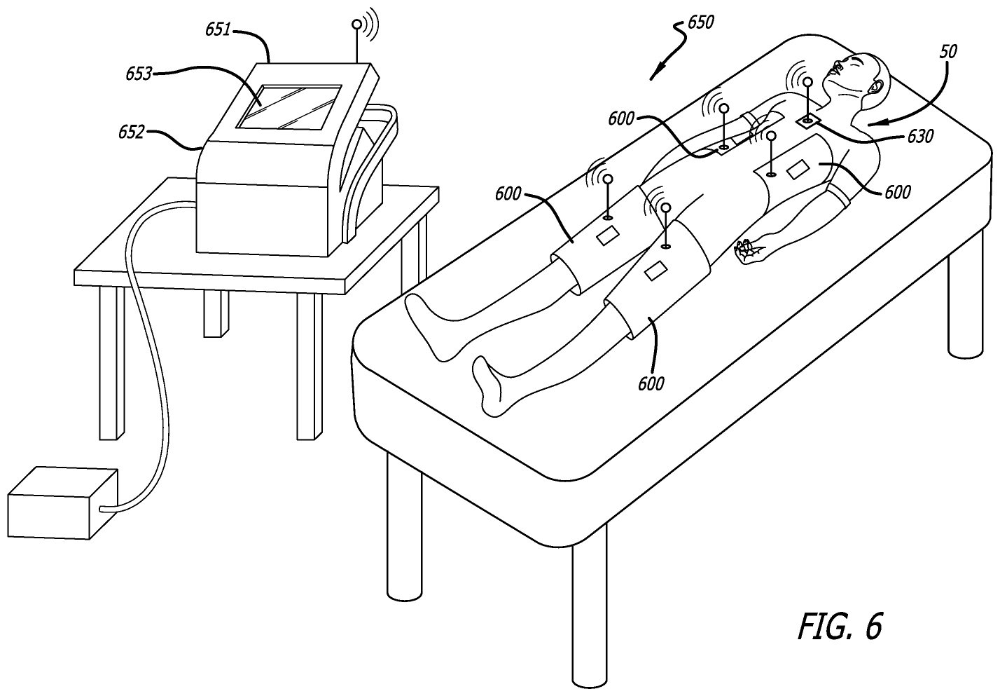

FIG. 6 illustrates a TTM system 650 comprising multiple thermal pads 600 coupled to the system control module 651. Each of the thermal pads 600 may be a thermal pad 100, a thermal pad 400, or a thermal pad 500 as described above. The thermal pads 600 may be coupled to the system control module 651 via a wired or a wireless connection. While the illustrated embodiment, comprises four thermal pads, other embodiments of the TTM system 650 may comprise one, two, three, five, or more thermal pads 600. The TTM system 650 may comprise a body temperature sensor 630 coupled to the system control module 651 via a wired or a wireless connection.

In some embodiments, the system control module 651 comprises an operator interface 653 included within a module housing 652. In some embodiments, the operator interface 653 may be a graphical user interface. The operator interface 653 may be configured to display information pertaining to the TTM therapy or the operation of the TTM system 650. The operator interface 653 may be further configured to provide for input from the clinician. As such, the clinician may control the operation of the TTM system 650 via the operator interface 653. The operator interface 653 may facilitate the setting of operating parameters of the TTM system 650 according to a prescribed TTM therapy. The operating parameters may include at least a target patient temperature and target pad temperatures for the thermal pads 600. The system control module 651 may comprise a console as further described below. In other embodiments, the system control module 651 may be a software module configured to operate on a network computer, a tablet, or a cell phone.

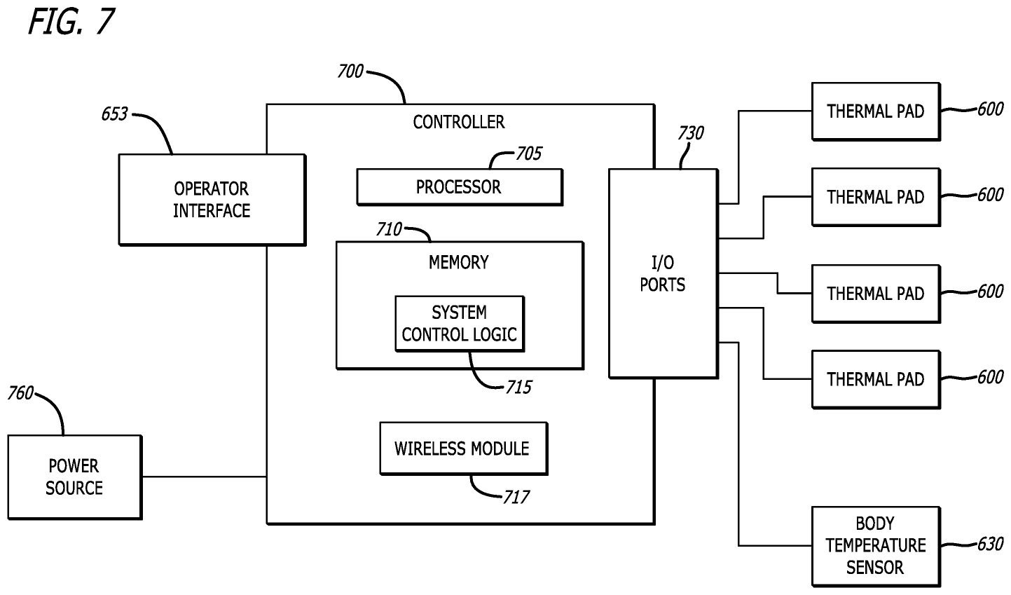

FIG. 7 illustrates a block diagram of a console 700 of the system control module 651 of FIG. 6. The console 700 comprises a processor 705 and memory 710 including a non-transitory, computer-readable storage medium, and the memory 710 includes TTM system control logic 715 stored thereon. The console 700 further includes a wireless module 717, and input/output (I/O) ports 730. The console 700 may comprise a power source 760 such as a battery. In other embodiments, the console 700 may receive electrical power from a facility power system. The I/O ports 730 facilitate connection of the thermal pads 600 and a body temperature sensor 630 via a wired connection. In some embodiments, coupling to the thermal pads 600 and the body temperature sensor 630 may be wireless, in which case the I/O ports 730 may be omitted. The console 700 is coupled to the operator interface 653 of the system control module 651.

The system control logic 715 may control the operation of the thermal pads 600 in accordance with the operating parameter settings. The system control logic 715 may communicate the target pad temperatures to each of the thermal pads 600 as may be defined by the parameter settings. The system control logic 715 may receive patient temperature data from the body temperature sensor 630 indicating the current temperature of the patient 50. The system control logic 715 may compare the patient temperature data with the target patient temperature, and as a result of the comparison, the system control logic 715 may modify the target pad temperatures and communicate revised target pad temperatures to the thermal pads 600. The system control logic 715 may further enable and/or disable operation of any or all of the thermal pads 600 according to a TTM therapy.

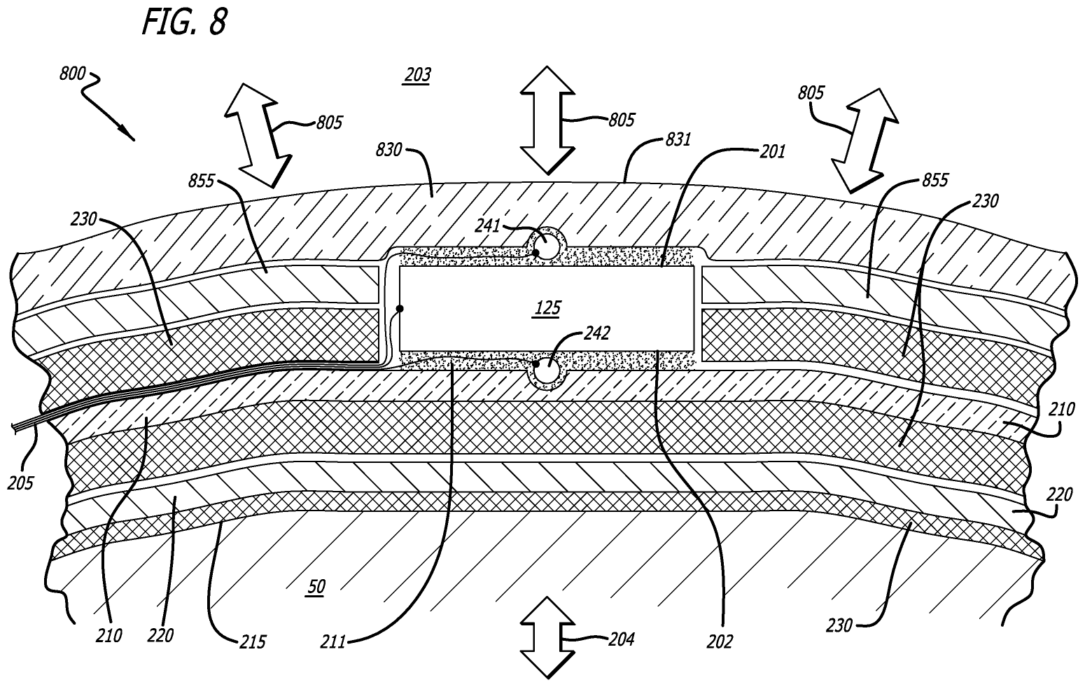

FIG. 8 is an illustration of a thermal pad 800 that can, in certain respects, resemble components of the thermal pad described in connection with FIGS. 1-7. It will be appreciated that all the illustrated embodiments may have analogous features. Accordingly, like features are designated with like reference numerals. Relevant disclosure set forth above regarding similarly identified features thus may not be repeated hereafter. Moreover, specific features of the thermal pad 100 and related components shown in FIGS. 1-7 may not be shown or identified by a reference numeral in the drawings or specifically discussed in the written description that follows. However, such features may clearly be the same, or substantially the same, as features depicted in other embodiments and/or described with respect to such embodiments. Accordingly, the relevant descriptions of such features apply equally to the features of the thermal pad 800 of FIG. 8. Any suitable combination of the features, and variations of the same, described with respect to the thermal pad 100 and components illustrated in FIGS. 1-7 can be employed with the thermal pad and components of FIG. 8, and vice versa. This pattern of disclosure applies equally to further embodiments depicted in subsequent figures and described hereafter.

The thermal pad 800 includes a top thermal conduction layer 830 disposed along a top surface of the insulation layer 855. The insulation layer 855 is configured to minimize thermal energy transfer between the thermal conduction gel 230 and the top thermal conduction layer 830. The top thermal conduction layer 830 is thermally coupled to the top side 201 of the TED 125. The top thermal conduction layer 830 may be formed of a metalized film (e.g., one or more layers of copper, aluminum and/or brass). The top thermal conduction layer 830 may be flexible so that the thermal pad 100 may acquire a curved shape when applied to the patient 50. In some embodiments, the top thermal conduction layer 830 may extend away from the TED 125 to the perimeter of the thermal pad 800 to define a convective surface 831. The convective surface 831 facilitates thermal energy transfer 805 from the top side 201 of the TED 125 to the environment 203 via natural convection.

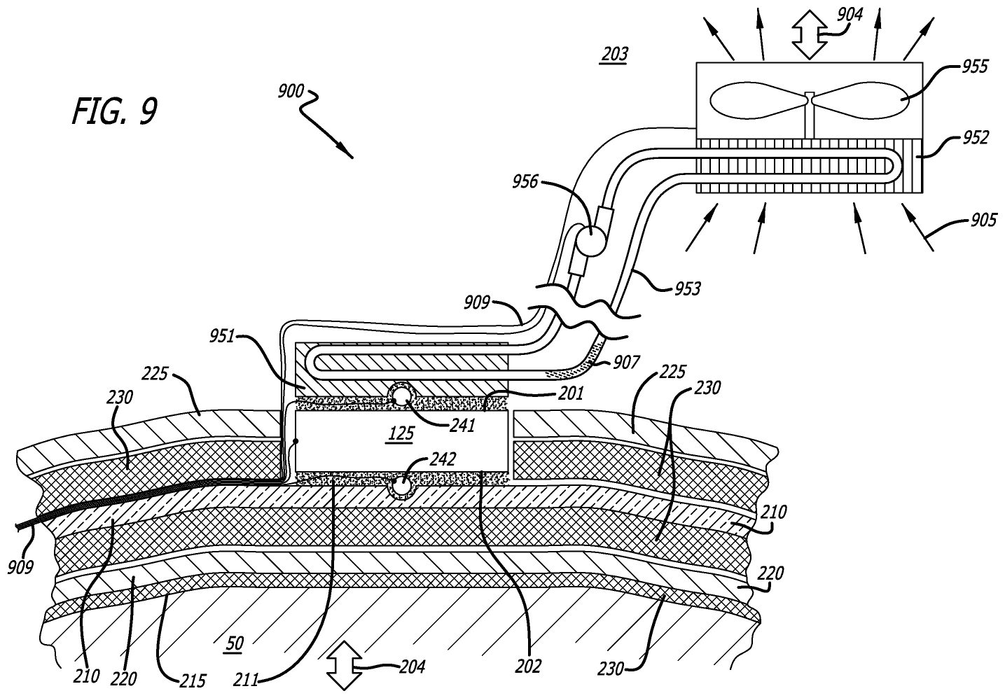

FIG. 9 illustrates a thermal pad 900, in accordance with some embodiments. The thermal pad 900 employs a process of circulating a liquid 907 between a heat exchanger (i.e., a first thermal convection device) 951 thermally coupled to the TED 125 and a radiator (i.e., a second thermal convection device) 952 disposed away from the TED 125. The thermal pad 900 includes a pump 956 to facilitate circulating flow of the liquid 907 between the heat exchanger 951 and the radiator 952 through the tubing 953. A fan 955 defines air flow 905 through the radiator 952 to transfer thermal energy 904 away from the liquid 907 to the environment 203. By way of summary, the top side 201 of the TED 125 conducts thermal energy to the heat exchanger 951 which transfers thermal energy to the liquid 907. The liquid 907 flows through the tubing 953 to the radiator 952 where the liquid 907 transfers thermal energy 904 to the air of the environment 203. The liquid 907 then flows back to the heat exchange 951 to receive thermal energy from the heat exchanger 951. In some embodiments, the radiator 952, together with the fan 955, may be located away from the patient 50 by a considerable distance, such as several feet. By locating the process of transferring thermal energy 904 to the environment 203 away from the patient 50, patient comfort and safety may be enhanced. In some embodiments, the fan 955 may be omitted so that air flows naturally through the radiator 952.

In some embodiments, the pump 956 and/or the fan 955 may be coupled to the power converter 320 of the controller 110 (FIG. 3) via the wires 909, and the temperature control logic 315 may be configured to control the pump 956 and/or the fan 955. As such, the temperature control logic 315 may be configured to control the temperature of the top side 201 of the TED 125 to establish or maintain a top-side temperature within a temperature range consistent with maximizing or optimizing an efficiency of the TED 125 or an efficiency of the thermal pad 900 as a whole. The temperature control logic 315 may receive temperature data from the top-side temperature sensor 241 and adjust an electrical power supplied to the fan 955 and/or the pump 956 to move the temperature of the top side 201 of the TED 125 toward a target top-side temperature stored in the memory 310. More specifically, the temperature control logic 315 may compare temperature data received from the top-side temperature sensor 241 with the target top-side temperature. As a result of the comparison, the temperature control logic 315 as executed by the processor 305 may cause the power converter 320 to increase or decrease the electrical power supplied to the fan 955 and/or the pump 956 to move the top-side temperature toward the target top-side temperature. By way of summary, the temperature control logic 315 may be configured to establish and maintain the top-side temperature at the target top-side temperature.

Without further elaboration, it is believed that one skilled in the art can use the preceding description to utilize the invention to its fullest extent. The claims and embodiments disclosed herein are to be construed as merely illustrative and exemplary, and not a limitation of the scope of the present disclosure in any way. It will be apparent to those having ordinary skill in the art, with the aid of the present disclosure, that changes may be made to the details of the above-described embodiments without departing from the underlying principles of the disclosure herein. In other words, various modifications and improvements of the embodiments specifically disclosed in the description above are within the scope of the appended claims. Moreover, the order of the steps or actions of the methods disclosed herein may be changed by those skilled in the art without departing from the scope of the present disclosure. In other words, unless a specific order of steps or actions is required for proper operation of the embodiment, the order or use of specific steps or actions may be modified. The scope of the invention is therefore defined by the following claims and their equivalents.

What is claimed is:

1. A medical pad for providing a targeted temperature management (TTM) therapy to a patient, the pad comprising:
at least one thermoelectric device (TED) having a top side and a bottom side, the TED configured to establish a temperature difference between the top side and the bottom side in accordance with electrical energy supplied to the TED;
a thermal conduction layer coupled to the bottom side; and
a skin contact layer coupled to the thermal conduction layer,
wherein the pad is configured to exchange thermal energy with the patient according to a temperature difference between the bottom-side temperature and a temperature of the patient.

2. The pad according to claim 1, wherein the pad is configured to extract thermal energy away from the patient.

3. The pad according to claim 1, further comprising a thermal conduction gel disposed across at least a portion of one of a top side or a bottom side of the thermal conduction layer.

4. The pad according to claim 1, further comprising a convection device coupled to the top side of the TED, wherein the convection device is configured to facilitate thermal energy exchange between the TED and environmental air adjacent the top side of the TED.

5. The pad according to claim 4, further comprising a fan configured to provide airflow across the convection device.

6. The pad according to claim 1, further comprising a pad controller configured to regulate a pad temperature, the pad temperature defined at least partially by the bottom-side temperature of the TED.

7. The pad according to claim 6, wherein the controller is electrically coupled to the TED, the controller configured to regulate the electrical energy supplied to the TED.

8. The pad according to claim 7, further comprising a temperature sensor operatively coupled to the bottom side of the TED, wherein the temperature sensor is coupled to the controller, and wherein the controller is configured to regulate the pad temperature as measured by the temperature sensor.

9. The pad according to claim 7, further comprising a plurality of TEDs coupled to the conduction layer, wherein the controller is configured to regulate the electrical energy supplied to the plurality of TEDs.

10. The pad according to claim 9, further comprising a hinge portion extending between adjacent TEDs, the hinge portion configured to facilitate pivotable displacement of each TED with respect to an adjacent TED.

11. The pad according to claim 7, further comprising two or more temperature zones, each zone comprising one or more TEDs, wherein the controller is configured to independently regulate the pad temperature of each temperature zone.

12. The pad according to claim 7, further comprising a plurality of controllers, wherein each controller is configured to regulate the electrical energy supplied to a single TED or a subset of the plurality of TEDs.

13. The pad according to claim 6, further comprising a top side temperature sensor operatively coupled to the top side of the TED, wherein:
the top side temperature sensor is coupled to the controller,
the fan is coupled to the controller, and
the controller is configured to adjust electrical power supplied to the fan in order to regulate a top side temperature of the TED as measured by the top side temperature sensor.

14. The pad according to claim 13, further comprising a circulating liquid system configured to transfer thermal energy away from the top side of the TED, the liquid system comprising:
a heat exchanger coupled to the top side;
a radiator coupled to the heat exchanger via a liquid conduit loop, the loop providing a liquid delivery flow path from the heat exchanger to the radiator and a liquid return flow path radiator to the heat exchanger; and
a pump in line with the liquid conduit loop, wherein the fan is coupled to the radiator.

15. A targeted temperature management (TTM) system, comprising:
a thermal pad comprising:
a thermoelectric device (TED) having a top side and a bottom side, the TED configured to establish a temperature difference between the top side and the bottom side in accordance with electrical energy supplied to the TED; and
a pad controller coupled to the TED, the pad controller configured to regulate a pad temperature of the thermal pad according to a target pad temperature stored in memory on the pad controller, the pad temperature defined at least partially by a bottom-side temperature of the TED; and
a system controller coupled to the thermal pad, wherein the system controller is configured to define the target pad temperature of the thermal pad.

16. The system according to claim 15, wherein the system controller is coupled to the thermal pad via a wireless connection.

17. The system according to claim 15, further comprising a patient temperature sensor configured to measure a temperature of the patient, the patient temperature sensor coupled to the system controller.

18. The system according to claim 15, wherein the system controller is configured to define the target pad temperature according to a target patient temperature stored in memory on the system controller.

19. The system according to claim 18, further comprising a plurality of thermal pads, wherein the system controller is configured to define the target pad temperature for each of the plurality of thermal pads.

20. The system according to claim 19, wherein the target pad temperature for one of the plurality of thermal pads is different from the target pad temperature for another of the plurality of thermal pads.

21. A method of providing a targeted temperature management (TTM) therapy to a patient, comprising:
providing a thermal pad comprising at least one thermoelectric device coupled to a controller;
applying the thermal pad to the patient;
coupling the thermal pad to an electrical power source; and
exchanging thermal energy between the patient and environmental air adjacent the pad, wherein the thermal energy passes through the pad.

22. The method according to claim 21, further comprising controlling a thermal pad temperature at a target pad temperature stored in memory on the controller.

23. The method according to claim 22, wherein the target pad temperature is lower than a temperature of the patient.

24. The method according to claim 22, wherein the pad comprises a first temperature zone and a second temperature zone, and wherein controlling a thermal pad temperature comprises:
controlling a first thermal pad temperature of the first temperature zone; and
controlling a second thermal pad temperature of the second temperature zone,
wherein the second thermal pad temperature is different from the first thermal pad temperature.

25. The method according to claim 22, wherein the thermal pad further comprises a patient temperature sensor coupled to the controller, and wherein controlling a thermal pad temperature comprises defining target pad temperature to achieve a target patient temperature stored in memory on the controller.
